(19) United States
(12) Patent Application Publication
(10) Pub. No.: US 2019/0359769 A1
(43) Pub. Date: Nov. 28, 2019

(54) PHARMACEUTICAL AGENT FOR IRON CHELATING

(71) Applicant: Johannes Gutenberg-Universität Mainz, Mainz (DE)

(72) Inventors: Holger FREY, Emmendingen (DE); Tobias JOHANN, Mainz (DE)

(21) Appl. No.: 16/477,836

(22) PCT Filed: Jan. 24, 2018
(86) PCT No.: PCT/EP2018/051709

§ 371 Date: Jul. 12, 2019

(30) Foreign Application Priority Data
Jan. 26, 2017 (DE) 10 2017 101 548.5

(51) Int. Cl. C08G 65/333; C08G 65/22; C08F 8/30; C08G 81/00; A61K 31/787

(52) U.S. Cl. CPC C08G 65/33396; C08G 65/22; A61K 31/787; C08G 81/00; C08F 8/30

(57) ABSTRACT

The invention relates to a pharmaceutical agent for the complexation of iron. The pharmaceutical agent includes an initiator group, a polymer and a terminal group R⁷, and has the structure: initiator group-polymer-R⁷. The pharmaceutical agent further includes one or more functional hydroxamic acid groups of the type —(C═O)NHOH or —(C═O)NCH₃OH.

The present invention relates to a pharmaceutical for the physiological complexation of iron in the event of a pathological iron overload.

Iron is an essential trace element for the human organism, and also for numerous other organisms. The effect of iron in the organism is based on the involvement of Fe²⁺ and Fe³⁺ ions in reduction and oxidation processes. However, Fe²⁺ ions are highly toxic and Fe³⁺ ions are water-insoluble at a physiological pH of 7.4. For this reason, most organisms, in the course of evolution, have developed complex mechanisms for the uptake, transport and storage of iron. For this purpose, microorganisms use high-affinity iron ligands or siderophores of low molecular weight, including enterobactins. Higher organisms, such as mammals, utilize specialized transport and storage proteins.

Under normal physiological conditions, the human body contains 3.5 to 5 g of iron, predominantly (˜70%) in the form of hemoglobin in erythrocytes and erythroid progenitor cells. The remaining about 30% of iron can be found in the myoglobin and intracellular stores such as the hepatocytes of the liver, spleen and bone marrow macrophages, and in proteins and enzymes involved in cellular respiration. In man, iron metabolism is largely conservative, and efficient recycling of iron present in hemoglobin makes a crucial contribution. Digestion also plays an important role for the homeostasis of iron in the human body, by means of which about 1 to 2 mg of iron are absorbed daily. Iron is excreted in about the same amount daily by the secretion of epithelial cells, skin and intestinal secretion, and small losses of blood in the digestive tract.

Under physiological conditions, iron is complexed by proteins including transferrin (Tf), and it is ensured that there is no formation of free radicals. Iron bound to transferrin is transported in the plasma and is not available for redox reactions. Transferrin has a high iron capacity and ensures that no free toxic, non-transferrin-bound iron (NTBI) is available. Transferrin contains 2 to 3 mg of iron and is saturated only to an extent of about 70% under normal physiological conditions. Transferrin transports iron to the hepatocytes and specific binding sites on the precursors of red cells of bone marrow that are involved in the synthesis of hemoglobin. In addition, transferrin binds iron which is released into the plasma by intestinal erythrocytes or by cells that catabolize senescent red blood cells.

Within the cells, ferritin is the main storage molecule for reusable iron with a fraction of about 27% (1 g) of the total amount of iron in the body. Ferritin has a storage capacity of 4500 atoms of iron per ferritin molecule and ensures that iron is in a redox-inactive form within the cell. Accordingly, ferritin reduces toxicity owing to formation of free radicals and at the same time keeps iron in mobile form for metabolic processes. In the event of oxidative stress, ferritin removes iron ions and oxygen from the cytoplasm and assists the return to normal redox conditions. In pathological states with “iron overloading”, excess iron in the form of insoluble “iron cores” composed of partly degraded ferritin or hemosiderin is excreted primarily in the liver, spleen, endocrine organs and cardiac muscle tissue. Even though electron shuttling is crucial for metabolic processes, excess iron can catalyze harmful reactions that generate free radicals and reactive oxygen species. These processes can proceed via the Haber-Weiss reaction in which hydrogen peroxide (H₂O₂) reacts with a superoxide radical (O₂) and forms the physiologically highly reactive hydroxyl radical (OH*). Even though this reaction takes place to a minimal degree under normal physiological conditions, it can be catalyzed by iron and lead to an accumulation of free radicals that interact with cellular components and impair metabolic functions. It is known that the increased formation of free radicals can oxidize lipids, proteins and DNA in organs, the heart being the most susceptible. Even in the event of a slight iron excess, the normal cellular redox equilibrium can be disrupted, and the amount of excess iron is crucial for the organ damage that results therefrom.

By contrast with the highly developed mechanisms for absorption, transport and storage of iron, for lack of any physiological pathway for the active excretion of iron, there is virtually no natural option for reduction of iron excess. This is found to be extremely problematic for the treatment of diseases such as β-thalassemia (β-TM), sickle-cell anemia (SCD) and myelodysplastic syndrome (MDS), which cause an iron overload. By means of transfusion of red blood cells, anemia is alleviated in patients suffering from β-TM and MDS, and vascular occlusions and strokes are prevented in SCD patients. Owing to significant impairment of erythropoiesis in the event of these diseases, the iron content in the plasma rises significantly with a conversion rate of 10 to 15 times the normal level. This results in an accumulation of excess iron of about 2.5 g per year. In addition to this intrinsic iron accumulation, the patients receive blood transfusions typically containing about 250 mg of iron at regular intervals. In order to keep the iron content in the plasma within the physiologically normal range, the patients are given accompanying treatment by means of iron chelation. Iron chelation is clinically indicated for patients suffering from β-TM, MDS or SCD and receiving blood transfusions. In the context of iron chelation, active ingredient molecules that bind iron under physiological conditions and form a non-toxic complex or a chelate which is subsequently excreted renally or fecally are used. Iron chelation protects cells from oxidative damage by reducing the content of reactive iron in the plasma and the cytosolic labile iron pool.

Three active ingredients are currently clinically approved for iron chelation: deferoxamine (DFO), deferiprone (DFP) and deferasirox (DFX).

Deferoxamine (DFO) is a hexadentate ligand having a molar mass MW=561 g·mol⁻¹. DFO is a high-affinity chelator for Fe³⁺ and forms very stable iron complexes with a logarithmic stability constant of 30. By means of DFO, it is possible to considerably extend the life expectancy of patients suffering from β-TM, MDS or SCD and receiving blood transfusions. Moreover, the incidence of heart damage, liver failure and endocrine disorders can be significantly reduced. In spite of its helpful action, DFO has considerable disadvantages. Owing to its low lipophilicity and high molar mass, DFO is absorbed only very slowly by gastrointestinal cells and has a short physiological half-life of only about 5 to 20 min. Accordingly, DFO has to be administered subcutaneously with a dose of 40-60 mg per kg of bodyweight, distributed over 8-12 h, 5-7 days per week. As a result of the cumbersome form of administration, patient compliance is inadequate. On the other hand, in the case of higher dosage of DFO, severe neurotoxic disorders, such as neurological loss of hearing, electroretinal anomalies, reduced bone development and growth disorders can occur.

Deferiprone (DFP) is a bidentate ligand having a molar mass MW=139 g·mol⁻¹ and, by contrast with DFO, is administered orally. Owing to doubts with regard to safety and chelation efficiency, clinical approval for DFP was granted in Europe and the USA only with a considerable delay. At a low concentration ratio of DFP to iron, iron ions are merely partially complexed by DFP. Enrichment of partially complexed iron ions can form redox potentials. Moreover, partially complexed iron ions can catalyze the formation of harmful free radicals and reactive oxygen species. In studies, inadequate lowering of the iron content was observed in multiple patients treated with DFP. In a long-term study, in the case of treatment with DFP, an elevated incidence of heart failure was found by comparison with DFO. A significant reason for the reduced efficiency of DFP is its rapid metabolization in the liver. The 3-hydroxy-functional group of DFP which is essential for the chelation of iron causes rapid metabolization in liver cells by glucoronidation. For instance, in one study, a proportion of 85% of the DFP dose administered was found in the urine in the form of inactive 3-O-glucoronide conjugates. Apart from its problematic metabolization, DFP has side effects such as agranulocytosis and neutropenia.

Deferasirox (DFX) is a tridentate ligand having a molar mass MW=373 g·mol⁻¹ and is likewise administered orally. DFX has high selectivity and affinity for Fe³⁺ without increasing the excretion of other metals such as zinc and copper. In studies on rats and humans, a physiological half-life of 8-16 h was observed. Iron excretion with DFX is about 5 times and 10 times higher than in the case of use of DFO and DFP respectively. DFX is lipophilic and has high cell permeability. Owing to its long physiological half-life and efficient iron excretion, treatment with DFX can be effected by means of an oral dose administered once daily. In spite of the many benefits, there are considerable concerns with regard to the long-term toxic effects of DFX. In various studies, renal toxicity, hepatic dysfunction and thrombocytopenia have been observed, as has an elevated incidence of Fanconi syndrome. In a more recent long-term study, elevated lethality compared to DFO and DFP was found in older MDS patients undergoing DFX treatment.

The aim of the present invention is to provide improved pharmaceuticals for iron chelation. For this purpose, well-defined macromolecular structures functionalized with hydroxamic acid are proposed. Only a few studies relating to hydroxamic acid-functionalized macromolecular structures are known to date. Almost all polymers having hydroxamic acid-functional groups were prepared by polymer-analogous reactions. The spectrum of structures is limited to polymers having a vinyl backbone, especially polymethylmethacrylates, polyacrylamides or specific NHS active ester polymers that have been prepared by free-radical polymerization. Complete functionalization is unknown to date. Most studies are application-specific and do not show any systematic concept for synthesis of defined polymeric structures (Domb, A.; Langer, R.; Cravalho, E.; Gershon, G.; Mathiowitz, E.; Laurencin, C.; Massachusetts Institute of Technology, Cambridge, Mass.; Method of making Hydroxamic Acid Polymers from Primary Annide Polymers U.S. Pat. No. 5,128,420 A; Winston, A.; Mazza, E. T. J. Polym. Sci. Part A: Polym. Chem. 1975, 13, 2019-2030; Kern, W.; Schulz, R. C. Angew. Chem. 1957, 69, 153-171).

Studies on direct free-radical polymerization of a hydroxamic acid-functional monomer showed the occurrence of side reactions with degradation of the hydroxamic acid (Iskander, G. M.; Kapfenstein, H. M.; Davis, T. P.; Wiley, D. E. J. Appl. Polym. Sci. 2000, 78, 751-758).

There is no method known to date for direct preparation of hydroxamic acid-functional polymers or polyethers. There are merely two studies relating to the functionalization of polyethers with hydroxamic acid groups. Kizhakkedathu et al., by the action of highly reactive reagents such as sodium periodate and sodium cyanoborohydride, functionalized hyperbranched polyglycerol (hbPG) and poly(oligoethylene glycol methacrylate) with deferoxamine, a naturally occurring trishydroxamic acid (Hamilton, J. L.; Kizhakkedathu, J. N. Mol. Cell. Ther. 2015, 3, 3; Rossi, N. A. A.; Mustafa, L.; Jackson, J. K.; Burt, H. M.; Horte, S. A.; Scott, M. D.; Kizhakkedathu, J. N. Biomaterials 2009, 30, 638-648; Imran ul-haq, M.; Hamilton, J. L.; Lai, B. F. L.; Shenoi, R. A.; Horte, S.; Constantinescu, I.; Leitch, H. A.; Kizhakkedathu, J. N. ACSNano 2013, 7, 10704-10716). Under these reaction conditions, many multifunctional structures are unstable, and exact control of the number of hydroxamic acids is impossible.

In general, hydroxamic acids are excellent complexing agents for medical and industrial applications (Codd, R. Coord. Chem. Rev. 2008, 252, 1387-1408). Polyethers, especially polyethylene glycol and derivatives thereof, are established in medical use and are obtainable with defined structure by anionic ring-opening polymerization. Particularly the low toxicity, the water solubility and the stealth effect are a great benefit for therapeutic purposes. The use of epoxy derivatives such as ethylene oxide, propylene oxide, butylene oxide and glycidyl ethers make it possible to prepare multifunctional polyethers. These give access to further reactions, for example click reactions for preparation of protein conjugates or specific properties such as LCST characteristics (Herzberger, J.; Niederer, K.; Pohlit, H.; Seiwert, J.; Worm, M.; Wurm, F. R.; Frey, H. Chem. Rev. 2016, 116 (4), 2170-2243; Dingels, C.; Schönner, M.; Frey, H. Chem. unserer Zeit 2011, 45, 338-349).

Many possible uses of polymeric complexing agents using catechol-functional polyethers have been described in the literature. Analogously to catechol-functional polymers, hydroxamic acid-functional polyethers could possibly be used as a surface coating with antifouling properties (Gillick, T.; Benetti, E. M.; Rakhmatullina, E.; Konradi, R.; Li, W.; Zhang, A.; Schlüter, A. D.; Textor, M. JACS 2011, 133, 10940-10950). Also known is the functionalization of nanoparticles for stabilization in aqueous solution by catechol-functional polyethers (Wilms, V. S.; Bauer, H.; Tonhauser, C.; Schümann, A.-M.; Müller, M.-C.; Tremel, W.; Frey, H. Biomacromolecules 2013, 14, 193-199; Niederer, K.; Schüll, C.; Leibig, D.; Johann, T.; Frey, H. Macromolecules 2016, 49, 1655-1665). These can be used as MRT contrast agents. Hydroxamic acid is an alternative to catechols which is less toxic and more stable with regard to oxidation.

Kizhakkedathu et al., using DFO-hbPG conjugates, showed the potential of hydroxamic acid-functional polyethers in medical use. Deferoxamine is on the list of essential medicines of the World Health Organization and is the most important treatment approach for prevention of lethal iron poisoning (Marmion, C. J.; Griffith, D.; Nolan, K. B. Eur. J. Inorg. Chem. 2004, 2004, 3003-3016; World Health Organization, Model List of Essential Medicines, 2015). For successful treatment, as a result of the short plasma half-life of about 5 minutes, subcutaneous injections have to be undertaken over several hours. Kizhakkedathu et al. showed that the conjugation of deferoxamine with hyperbranched polyglycerol increased the half-life to up to 44 hours.

In a departure from the known approaches, hydroxamic acid-functional polyethers represent an alternative to deferoxamine, deferiprone and deferasirox with elevated plasma half-life and/or reduced toxicity. This would mean a significant improvement in treatment for patients suffering from β-TM, SCD or MDS and reliant on lifelong treatment with DFO, DFP or DFX.

The use of the 1,4,2-dioxazole group as protected hydroxamic acid for synthesis of polymers was unknown to date and is proposed for the first time for this use in the present invention. 1,4,2-Dioxazole-protected hydroxamic acid derivatives offer an approach for the systematic preparation of polymers by which multifunctional polymer architectures are also obtainable without polymer-analogous reactions. By contrast with polymer-analogous reactions, it is possible to introduce a defined number of hydroxamic acid groups into the polymer. This counteracts crosslinking of the polymer molecules by multiple functional groups per polymer molecule. On the basis of the concept of the invention, a multitude of structures is accessible, which open up new possibilities both in medicine and for industrial use. The present invention for the first time describes direct anionic ring-opening polymerization proceeding from protected hydroxamic acid-functional initiators and protected hydroxamic acid-functional epoxy monomers.

It is an object of the present invention to provide a process and a pharmaceutical produced by the process for physiological iron chelation and excretion, having an elevated physiological half-life and/or reduced toxicity compared to known active ingredients, such as DFO, DFP and DFX.

This object is achieved by a process comprising the steps of
(a) providing an initiator selected from the group comprising
   alcohols, for example HOCH₃, HOCH₂CH₃, HO(CHCH₃)CH₃, HO(CH₂)₂CH₃, HO(CH₂)₃CH₃, HO(CH₂)₄CH₃;
   compounds containing a protected hydroxamic acid group of the type

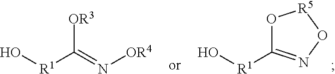

and
lithium organyls and free-radical initiators, for example n-butyllithium, sec-butyllithium, dibenzoyl peroxide, azoisobutyronitrile, potassium peroxodisulfate, ammonium peroxodisulfate;
(b) providing monomers selected from the group comprising
   epoxides of the type

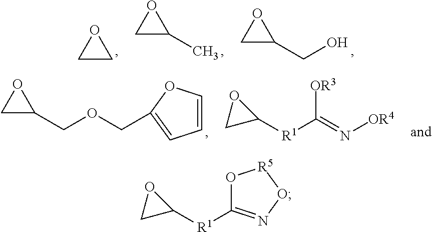

or
acrylics of the type

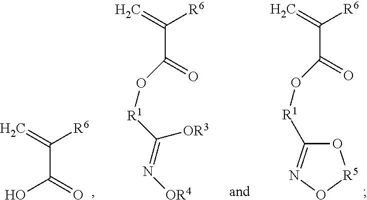

or
styrenes of the type

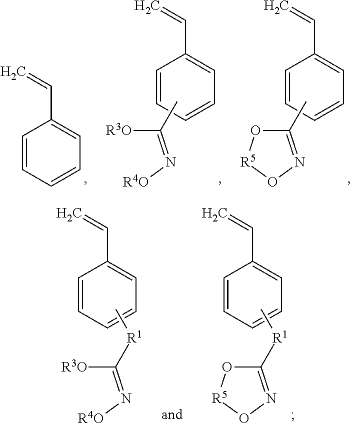

where
the initiator and/or one of the monomers contains a protected hydroxamic acid group or the at least one monomer is an epoxide including the epoxide

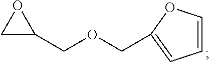 [SMILES: c1coc(COCC2CO2)c1]

R¹ is selected from
aliphatic groups of the —(CH₂)ₚ— type with p=1, 2, 3, 4, 5, 6, 7, 8, 9 or 10;
alkoxide groups, for example —OCH₂—, —OCH₂CH₂—, —O(CHCH₃)CH₂—, —O(CH₂)₃—, —O(CH₂)₄—, —O(CH₂)₃—;
aliphatic ether groups of the —(CH₂)_{q}O(CH₂)ₛ— type with q=1, 2, 3, 4, 5, 6, 7, 8, 9 or 10 and s=1, 2, 3, 4, 5, 6, 7, 8, 9 or 10;
oligoethylene glycol groups of the —(CH₂CH₂O)ₜ— type with t=1, 2, 3, 4, 5, 6, 7, 8, 9 or 10;
aromatic groups, such as phenol or naphthyl radicals; and
derivatives of the above groups;
R³ is a protecting group selected from the group comprising aliphatics —CₖH₂ₖ₊₁ with k=1, 2, 3, 4, 5, 6, 7, 8, 9 or 10, vinyl, allyl, phenyl, benzyl and silyls, such as trimethylsilyl and triisopropylsilyl;
R⁴ is a protecting group selected from the group comprising aliphatics —CₘH₂ₘ₊₁ with m=1, 2, 3, 4, 5, 6, 7, 8, 9 or 10, vinyl, allyl, phenyl, benzyl and silyls, such as trimethylsilyl and triisopropylsilyl;
R⁵ is selected from the group comprising ═(CH₂); acetonides, such as ═(C(CH₃)₂), ═(CHPh); cyclohexanone radicals, such as ═(C₆H₁₀); sodium tetraborate radical ═(B₄O₇);
R⁶ is selected from —H and —CH₃;
(c) mixing one or more of the monomers provided in step (b) with the initiator in a defined molar ratio; and
(d) polymerizing.

Advantageous embodiments of the process are characterized in that
the initiator has the structure

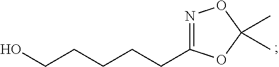

the initiator has the structure

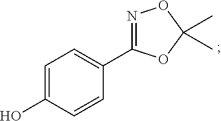

the initiator has the structure

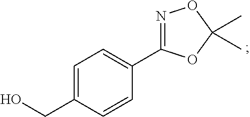

the initiator has the structure

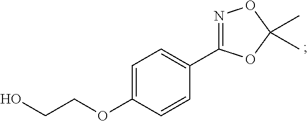 [SMILES: CC1(C)ON=C(c2ccc(OCCO)cc2)O1]

in step (a) the initiator is provided as a salt of one of the above compounds containing a protected hydroxamic acid group;
a monomer used in step (c) has the structure

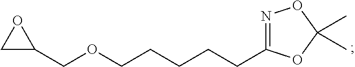 [SMILES: CC1(C)ON=C(CCCCCOCC2CO2)O1]

a monomer used in step (c) has the structure

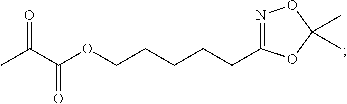 [SMILES: CC(=O)C(=O)OCCCCCC1=NOC(C)(C)O1]

a monomer used in step (c) has the structure

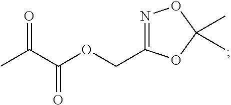

a monomer used in step (c) has the structure

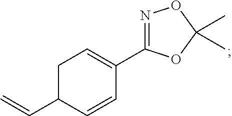

in step (c) the initiator and one or more of the monomers provided in step (b) are mixed in a molar ratio of initiator to monomer(s) (initiator:monomer(s)) in the range from 1:3 to 1:400, 1:3 to 1:300, 1:3 to 1:200 or 1:3 to 1:100;
in step (c) the initiator and one or more of the monomers provided in step (b) are mixed at a temperature of ≤0° C., ≤−10° C., ≤−20° C., ≤−30° C. or ≤−40° C.;
in step (c) the initiator and one or more of the monomers provided in step (b) are mixed in one or more solvents;
in step (c) the initiator and one or more of the monomers provided in step (b) are mixed in one or more organic solvents, such as hexane, benzene, toluene, tetrahydrofuran, dioxane, dimethyl sulfoxide;
in step (d) the polymerization is initiated by increasing the temperature of the reaction mixture obtained in step (c) to a temperature of ≥10° C., ≥20° C., ≥30° C., ≥40° C., ≥50° C. or ≥60° C.;
step (d) is executed at a temperature of ≥10° C., ≥20° C., ≥30° C., ≥40° C., ≥50° C. or ≥60° C.;
in a subsequent step (e) a further monomer provided in step (b) is added to the reaction mixture and the polymerization is continued;
step (e) is executed repeatedly;
the polymerization is ended by consumption of the at least one monomer or by addition of a terminator;
the polymerization is ended by adding a terminator selected from the group comprising protic reagents, such as H₂O; alcohols, such as methanol, ethanol, propanol; alkyl halides, such as methyl iodide, ethyl bromide, allyl chloride, allyl bromide, propargyl bromide; active esters; or activated carbonyl compounds, such as acid chlorides, acid anhydrides, N-hydroxysuccinimide ester;
in step (c) or (e) one or more of the epoxides provided in step (b) including the epoxide

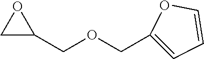 [SMILES: c1coc(COCC2CO2)c1]

are used and, after termination of the polymerization, a hydroxamic acid-functionalized compound of the type

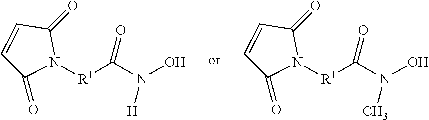

is added and conjugated with the furan groups of the polymer;
after termination of the polymerization the at least one hydroxamic acid group is deprotected; and/or
after termination of the polymerization the at least one hydroxamic acid group is deprotected by adding a deprotecting agent selected from the group comprising aqueous and nonaqueous solutions of inorganic acids, such as hydrochloric acid and sulfuric acid; aqueous and nonaqueous solutions of organic acids, such as para-toluenesulfonic acid and camphor-10-sulfonic acid; or by using acidic ion exchangers.

Preferably, all process steps are executed under standard conditions at room temperature, i.e. within a range from 20 to 35° C. and a pressure of 0.9 to 1.1 bar. In individual cases, however, it may be appropriate to execute some of the process steps at elevated or reduced temperature and/or elevated or reduced pressure.

In general, the polymerization in step (d) proceeds spontaneously, with a long polymerization time compared to the time which is required for the production of a homogeneous mixture of the monomers and the initiator by means of customary mechanical methods, such as stirring or pivoting in step (c) and optionally in step (e).

In exceptional cases in which the polymerization proceeds very quickly, step (c) is conducted at reduced temperature in order to assure a homogeneous mixture of the monomers and the initiator and then the temperature is increased in step (d) in order to initiate the polymerization.

In addition, in individual cases, in the case of a very long polymerization time, for the purpose of accelerating the reaction, step (d) is executed at elevated temperature.

The invention embraces pharmaceuticals preparable by a process comprising one or more of the steps described above.

The invention further provides a pharmaceutical for physiological iron chelation and excretion, which consists of an initiator group, a polymer and an end group R⁷, has the initiator group-polymer-R⁷ structure and comprises one or more functional hydroxamic acid groups of the —(C═O)NHOH or —(C═O)NCH₃OH type; where

the initiator group is selected from the group comprising
alkoxide groups, for example —OCH₃, —OCH₂CH₃, —O(CHCH₃)CH₃, —O(CH₂)₂CH₃, —O(CH₂)₃CH₃, —O(CH₂)₄CH₃;
hydroxamic acid-functionalized groups of the —R¹(C═O)NHOH or —R¹(C═O)NCH₃OH type; and
residues of a lithium organyl or free-radical initiator, for example CH₃(CH₂)₃—, CH₃CH₂(CHCH₃)—, Ph(C═O)O—, CNCH₃CH₃C—, SO₂OHO—;

the polymer consists of units selected from the group comprising

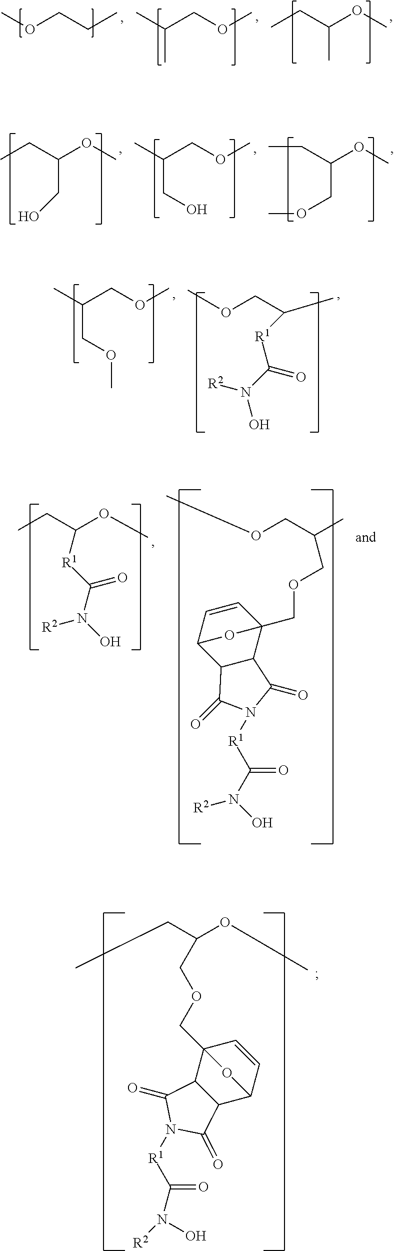

or

the polymer consists of acrylic units selected from the group comprising

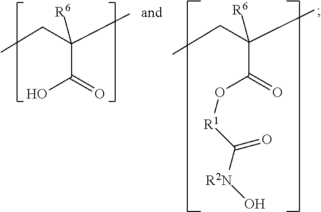

or

the polymer consists of styrene units selected from the group comprising

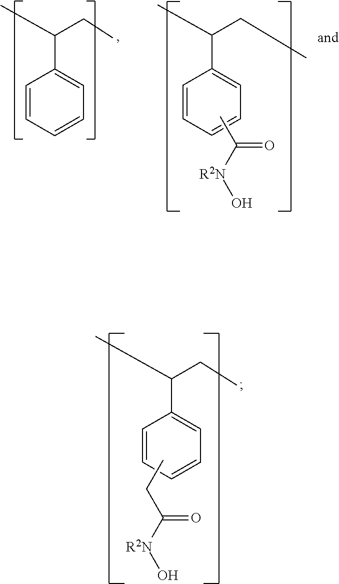

where

R¹ is selected from
aliphatic groups of the —(CH₂)ₚ— type with p=1, 2, 3, 4, 5, 6, 7, 8, 9 or 10;
alkoxide groups, for example —OCH₂—, —OCH₂CH₂—, —O(CHCH₃)CH₂—, —O(CH₂)₃—, —O(CH₂)₄—, —O(CH₂)₅—;
aliphatic ether groups of the —(CH₂)_{q}O(CH₂)ₛ— type with q=1, 2, 3, 4, 5, 6, 7, 8, 9 or 10 and s=1, 2, 3, 4, 5, 6, 7, 8, 9 or 10;
oligoethylene glycol groups of the —(CH₂CH₂O)ₜ— type with t=1, 2, 3, 4, 5, 6, 7, 8, 9 or 10;
aromatic groups, such as phenol or naphthyl radicals; and
derivatives of the above groups;

R² is selected from —H and —CH₃;

R⁶ is selected from —H and —CH₃;

R⁷ is selected from the group comprising —H; —CH₃; —(CH₂)ᵤCH₃ with u=1, 2, 3, 4, 5, 6, 7, 8, 9 or 10; esters, such as —(C═O)CH₃; allyl radicals; propargyl radicals; alcohol radicals, such as —OCH₃; —OCH₂CH₃; —OCH(CH₃)₂; —O(CH₂)₂CH₃.

Advantageous embodiments of the pharmaceutical are characterized in that
R¹ is a pentanol group —O(CH₂)₅—;
R¹ is a phenol group —O(C₆H₄)—;
R² is —H;
R² is —CH₃;
R⁶ is —H;
R⁶ is —CH₃;
the pharmaceutical has a structure of the OHNH(C═O)R¹-polymer-R⁷ or OHNCH₃(C═O)R¹-polymer-R⁷ type, where the polymer is a polyethylene glycol —(CH₂CH₂O)ₙ— with 3 n 100;
the pharmaceutical has a structure of the OHNH(C═O)R¹-polymer-R⁷ or OHNCH₃(C═O)R¹-polymer-R⁷ type, where the polymer is a polypropylene glycol —((CHCH₃)CH₂O)ₙ— with 3 n 100;
the pharmaceutical has a structure of the OHNH(C═O)R¹-polymer-R⁷ or OHNCH₃(C═O)R¹-polymer-R⁷ type, where the polymer is a linear polyglycerol —((CHCH₂OH)CH₂O)ₙ— with 3 n 100;
the pharmaceutical has a structure of the OHNH(C═O)R¹-polymer-R⁷ or OHNCH₃(C═O)R¹-polymer-R⁷ type, where the polymer is a branched polyglycerol consisting of 3 to 100 units, selected from the group comprising

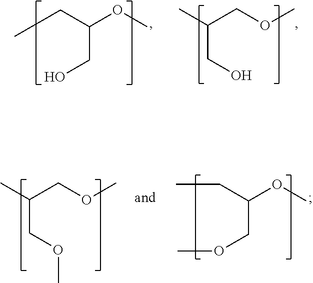

the pharmaceutical has a polydispersity M̅_{w}/M̅ₘ≤2;
the pharmaceutical has a polydispersity M̅_{w}/M̅ₙ≤1.6, preferably M̅_{w}/M̅ₙ≤1.2 and especially M̅_{w}/M̅ₙ≤1.1;
the pharmaceutical has a molar mass MW with 100 g·mol⁻¹≤MW≤2000 g·mol⁻¹;
the pharmaceutical has a molar mass MW with 100 g mol⁻¹≤MW≤600 g·mol⁻¹, 100 g·mol⁻¹≤MW≤400 g·mol⁻¹ or 100 g·mol⁻¹≤MW≤300 g·mol⁻¹;
the pharmaceutical has a molar mass MW with 600 g·mol⁻¹≤MW≤40 000 g·mol⁻¹;
the pharmaceutical has a molar mass MW with 800 g·mol⁻¹≤MW≤40 000 g·mol⁻¹, preferably 1000 g·mol⁻¹≤MW≤40 000 g mol⁻¹; and/or
the pharmaceutical has a molar mass MW with 600 g·mol⁻¹≤MW≤2000 g·mol⁻¹, 800 g·mol⁻¹≤MW≤2000 g·mol⁻¹, preferably 1000 g≤MW≤2000 g·mol⁻¹.

The invention further relates to the use of the above-described pharmaceuticals for treatment of pathological iron overload.

The invention is elucidated in detail hereinafter by examples.

Test Methods

All chemicals and solvents, unless listed separately, were sourced from the commercial suppliers (Acros, Sigma-Aldrich, Fischer Scientific, Fluka, Riedel-de Haen, Roth) and used without further purification. Deuterated solvents were sourced from Deutero GmbH (Kastellaun, Germany). Unless stated separately, all experiments were conducted at room temperature (20-25° C.), standard pressure (985-1010 hPa) and typical air humidity (40-100% RH) (source: measurement station at Institute for Atmospheric Physics, Johannes Gutenberg

University of Mainz).

NMR Spectroscopy

¹H and ¹³C NMR spectra were recorded on an Avance III HD 300 (300 MHz, 5 mm BBFO head with z gradient and ATM) from the manufacturer Bruker having a frequency of 300 MHz (¹H) or 75 MHz (¹³C). Spectra at 400 MHz (¹H) were recorded on an Avance II 400 (400 MHz, 5 mm BBFO head with z gradient and ATM) from the manufacturer Bruker. The chemical shifts are reported in ppm and are based on the proton signal of the deuterated solvent.

Gel Permeation Chromatography (GPC)

The GPC measurements were conducted according to DIN 55672-3 2016-01 with dimethylformamide (DMF), spiked with 1 g/L lithium bromide, as eluent on an instrument from the Agilent 1100 series with a HEMA 300/100/40 column from MZ-Analysetechnik. The signals were detected by means of an RI detector (Agilent G1362A) and UV (254 nm) detector (Agilent G1314A). For the recording of the GPC rate or curves, primarily the signal from the RI detector and if appropriate the signal from the UV detector was used. The measurements were conducted at 50° C. and a flow rate of 1.0 mL/min. Calibration was effected with polyethylene glycol 200, 1000, 2000, 6000, 20 000 and 40 000 standards and polystyrene standards from Polymer Standards Service.

Abbreviations:

PE petroleum ether

EA ethyl acetate

DCM dichloromethane

CSA DL-camphor-10-sulfonic acid

DMP 2,2-dimethoxypropane

eq equivalents

EO ethylene oxide

PO propylene oxide

### EXAMPLE 1: PREPARATION OF INITIATOR AND MONOMER

### (i) Synthesis of N,6-dihydroxyhexanamide

A 250 mL round-bottom flask is initially charged with 17.12 g (150 mmol, 1 eq) of epsilon-caprolactone, and a prepared solution of hydroxylamine in methanol is added. To prepare the hydroxylamine solution, 11.47 g (165 mmol, 1.1 eq) of hydroxylamine hydrochloride in 60 mL of methanol are neutralized with a solution of 12.62 g (225 mmol, 1.5 eq) of KOH in 32 mL of methanol, cooled and then filtered. After stirring at room temperature for 90 minutes, the reaction solution is acidified to pH 4 with concentrated HCl, concentrated in vacuo and recrystallized from THF. The product obtained after hot filtration and crystallization at −20° C. for h is 7.99 g (54 mmol, 36% of theory) of N,6-dihydroxyhexanamide as a colorless solid.

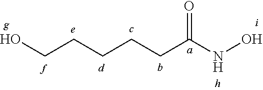

¹H NMR (300 MHz, DMSO-d6): [ppm]=10.37 (s, 0.9H, h), 9.72 (s, 0.1H, h), 9.03 (s, 0.1H, i), 8.68 (s, 0.9H, i), 4.37 (s, 1H, g), 3.35 (t, J=6.4 Hz, 2H, f), 1.93 (t, J=7.5 Hz, 2H, b), 1.52-1.42 (m, 2H, c), 1.42-1.32 (m, 2H, e), 1.30-1.16 (m, 2H, d).

¹³C NMR (75 MHz, DMSO-d6): [ppm]=169.25 (A), 60.67 (F), 32.42 (B), 32.30 (E), 25.23 (D), 25.16 (C).

IR-ATR [cm⁻¹]=3311 m, 3205 m, 3029 m, 2932 m, 2858 m br, 1616 vs (C═O), 1544 m, 1499 m, 1466 m, 1362 m, 1346 m, 1274 m, 1116 m, 1078 m, 1055 vs, 1012 vs, 977 vs, 785 m.

### (ii) Synthesis of the initiator 5-(5,5-dimethyl-1,4,2-dioxazol-3-yl)pentan-1-ol

A 500 mL flask is initially charged with 4.27 g (29 mmol, 1 eq) of N,6-dihydroxyhexanamide, which were suspended with 300 mL of dry dichloromethane. Subsequently, 8.90 g (67 mmol, 2.3 eq) of 2,2-diethoxy-propane and 6.74 g (29 mmol, 1 eq) of camphor-10-sulfonic acid are added and the mixture is stirred vigorously at room temperature. As soon as no reactant is detectable any longer (30 to 90 minutes), the reaction is admixed with 70 mL of saturated sodium hydrogencarbonate solution up to a pH of 8. The organic phase is removed and washed with 20 mL of saturated sodium hydrogencarbonate solution, dried over sodium sulfate and concentrated in vacuo. After purification by column chromatography by means of silica (petroleum ether:ethyl acetate 9:1->1:1), 1.25 g (7 mmol, 23% of theory) of 5-(5,5-dimethyl-1,4,2-dioxazol-3-yl)pentan-1-ol are obtained.

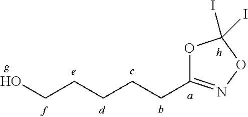 [SMILES: OCCCCCC1=NOC(I)(I)O1]

¹H NMR (300 MHz, chloroform-d): [ppm]=3.64 (t, J=6.3 Hz, 2H, f), 2.31 (t, J=7.3 Hz, 2H, b), 1.76 (s, 1H, g), 1.69-1.56 (m, 4H, e+c), 1.55 (s, 6H, i), 1.50-1.39 (m, 2H, d).

¹³C NMR (75 MHz, chloroform-d): [ppm]=160.39 (A), 114.53 (H), 62.65 (F), 32.26 (E), 25.22 (D), 25.18 (C), 24.91 (I), 23.95 (B).

### (iii) Synthesis of the monomer 5,5-dimethyl-3-(5-(oxiran-2-ylmethoxy)pentyl)-1,4,2-dioxazole

A 25 mL flask is initially charged with 210 mg (0.65 mmol, 0.1 eq) of tetrabutylammonium bromide, 1.217 g (6.5 mmol, 1 eq) of 5-(5,5-dimethyl-1,4,2-dioxazol-3-yl)pentan-1-ol and 6 mL of benzene, and 6 mL of 30% sodium hydroxide solution are added. Subsequently, 1.200 g (13.0 mmol, 2 eq) of epichlorohydrin are added gradually and the reaction mixture is stirred at room temperature for 48 h. The reaction solution is taken up in 100 mL of diethyl ether, and the organic phase is removed and washed twice with saturated sodium hydrogencarbonate solution, water and saturated sodium chloride solution, dried over sodium sulfate and concentrated in vacuo. After purification by column chromatography by means of silica (petroleum ether:ethyl acetate 4:1), 306 mg (1.3 mmol, 19% of theory) of 5,5-dimethyl-3-(5-(oxiran-2-ylmethoxy)pentyl)-1,4,2-dioxazole are obtained.

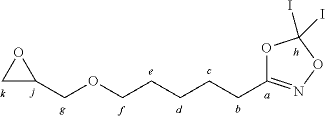 [SMILES: IC1(I)ON=C(CCCCCOCC2CO2)O1]

¹H NMR (300 MHz, DMSO-d6): [ppm]=3.66 (dd, J=11.5, 2.7 Hz, 1H, g), 3.41 (td, J=6.4, 1.2 Hz, 2H, f), 3.21 (dd, J=11.5, 6.4 Hz, 1H, g), 3.13-3.01 (m, 1H, j), 2.71 (dd, J=5.2, 4.2 Hz, 1H, k), 2.53 (dd, J=5.2, 2.7 Hz, 1H, k), 2.29 (t, J=7.3 Hz, 2H, b), 1.61-1.42 (m, 4H, e+c), 1.49 (s, 6H, i), 1.43-1.24 (m, 2H, d).

¹³C NMR (75 MHz, DMSO-d6); d [ppm]=159.64 (A), 113.84 (H), 71.19 (G), 70.24 (F), 50.34 (J), 43.39 (K), 28.67 (E), 24.93 (D), 24.65 (C), 24.39 (I), 22.95 (B).

IR-ATR [cm⁻¹]=2990 w, 2936 m, 2865 w, 1641 m (C═N), 1456 w, 1374 s, 1340 w, 1217 vs (C—O), 1153 m, 1110 vs, 1000 vs, 910 m, 850 m, 814 m, 760 m

Rf (EA:PE=1:1)=0.63

ESI-MS=244.1 (M+H, 100%), 260.2 (M+Na, 73%), 282.1 (M+K, 24%), 509.3 (2M+Na, 52%).

### EXAMPLE 2: SYNTHESIS OF ALPHA-1,4,2-DIOXAZOLE-FUNCTIONAL POLYETHYLENE GLYCOL

### (i) Polymerization of Polyethylene Glycol with alpha-1,4,2-dioxazole

A dried flask is initially charged with the initiator 5-(5,5-dimethyl-1,4,2-dioxazol-3-yl)pentan-1-ol and stirred with 0.9 eq of cesium hydroxide monohydrate and 5 mL of benzene under static vacuum for 60 minutes. Subsequently, the initiator salt is dried under high vacuum overnight and, the next day, dissolved in 10 mL of dry THF. Subsequently, ethylene oxide is cryotransferred into the reaction vessel and the reaction solution is stirred at 40-60° C. for 24 to 48 hours. After termination by addition of 1 mL of methanol, any solvent is removed in vacuo and the polymer is obtained by precipitation in ice-cold diethyl ether.

### (ii) Detachment of the Protecting Group and Deprotection of the Hydroxamic Acid

The alpha-1,4,2-dioxazole-functional polyethylene glycol is mixed with the same amount of DOWES 50WX8 ion exchange resin in isopropanol and agitated for 20 h. Subsequently, the solution is filtered, the filtrate is concentrated in vacuo and the hydroxamic acid-functional polyethylene glycol is obtained by precipitation in ice-cold diethyl ether.

### EXAMPLE 3: DETECTION AND QUANTIFICATION OF COMPLEXATION

The colorless hydroxamic acids form highly colored complexes with metals. It is usually trishydroxamatometal complexes that

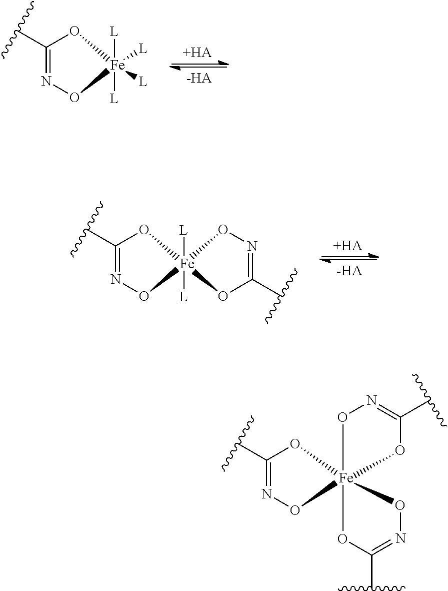

are formed.

(HA=hydroxamic acid)

In the case of iron(III), the result is an octahedral deep blue complex having an absorption maximum around 540 nm. By titration of a polymer solution with an iron(III) solution of known concentration, it is possible to photometrically monitor and quantify the formation of the complex. Formation of the mono complex is followed by the formation of the bis- and then of the trishydroxamatoiron(III) complex with constantly increasing absorption until occurrence of an asymptotic progression on total binding of the hydroxamic acids to the iron(III) atoms. FIG. 1 shows the absorption as a function of the concentration ratio of Fe³⁺ to the alpha-1,4,2-dioxazole-functional polyethylene glycol, which is identified in the diagram of FIG. 1 by HA for hydroxamic acid.

### EXAMPLE 4: STYRENE MONOMER WITH PROTECTED HYDROXAMIC ACID

### 1st Stage N-hydroxy-p-methylbenzamide

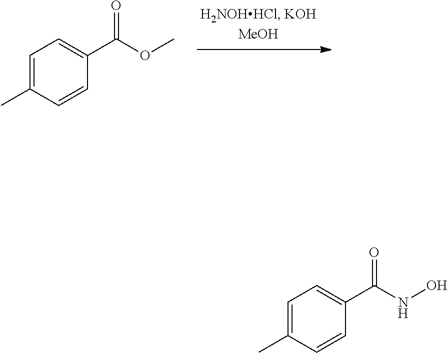

24.00 g (0.16 mol, 1 eq) of methyl 4-methylbenzoate and 33.32 g (0.48 mol, 3 eq) of hydroxylamine hydrochloride are dissolved in 500 mL of methanol. To this solution are added 53.86 g (0.96 mol, 6 eq) of powdered KOH in multiple portions. After 12 to 24 h, the precipitate is filtered off and washed with 200 mL of methanol, and the mother liquor is acidified to pH 4.0 with concentrated HCl. The solution is fully concentrated under reduced pressure. The residue is extracted 4 times with 150 mL each time of boiling THF, and the combined organic phase is dried over Na₂SO₄ and completely freed of solvents under reduced pressure. The residue is recrystallized from 250 mL of ethyl acetate. After 48 h at −20° C., 19.2 g (0.127 mol, 79% of theory) of N-hydroxy-p-methylbenzamide were obtained in the form of colorless platelets.

¹H NMR (400 MHz, DMSO-d₆) δ=10.95 (s, 1H), 9.23 (s, 1H), 7.72-7.51 (m, 2H), 7.31-7.15 (m, 2H), 2.33 (s, 3H).

### 2nd Stage 5,5-dimethyl-3-(p-tolyl)-1,4,2-dioxazole

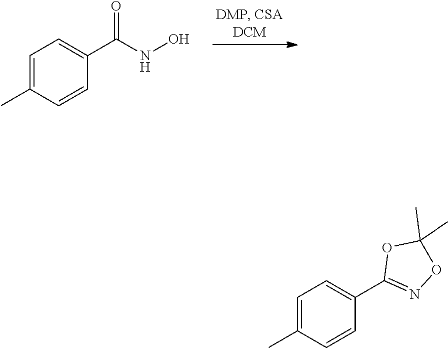

10.00 g (66.2 mmol, 1 eq) of N-hydroxy-p-methylbenz-amide are suspended in 1350 mL of dichloromethane, and 24.3 mL (20.67 g, 198.6 mmol, 3 eq) of 2,2-dimethoxy-propane (DMP) are added. Added to this suspension are 15.36 g (66.02 mmol, 1 eq) of DL-camphor-10-sulfonic acid (CSA) in one addition, and the resultant solution is stirred at room temperature for 3 hours. Subsequently, the reaction is ended by adding 200 mL of saturated NaHCO₃ solution. The organic phase is removed and the aqueous phase is extracted twice with 100 mL each time of dichloromethane. The combined organic phases are washed with 100 mL of saturated NaHCO₃ solution and 100 mL of saturated NaCl solution and dried over CaCl₂. After the solvent had been removed under reduced pressure, 11.34 g (59.3 mmol, 90% of theory) of 5,5-dimethyl-3-(p-tolyl)-1,4,2-dioxazole were obtained as a colorless liquid, which crystallizes gradually at −20° C. to give a colorless solid.

¹H NMR (400 MHz, chloroform-d) δ=7.69-7.64 (m, 2H), 7.25-7.18 (m, 2H), 2.39 (s, 3H), 1.67 (s, 6H).

### 3rd Stage 3-(4-(bromomethyl)phenyl)-5,5-dimethyl-1,4,2-dioxazole

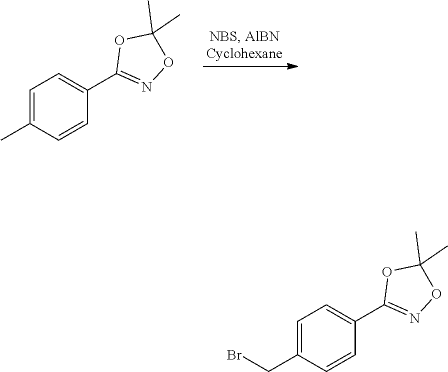

11.34 g (59.3 mmol, 1 eq) of 5,5-dimethyl-3-(p-tolyl)-1,4,2-dioxazole, 10.57 g (59.3 mmol, 1 eq) of N-bromosuccinimide (NBS) and 0.39 g (2.38 mmol, 0.04 eq) of azobisisobutyronitrile (AIBN) are suspended in 200 mL of dried and degassed (distilled over sodium) cyclohexane. The suspension is boiled under reflux under protective gas (argon) in an oil bath at 120° C. until the solution clarifies, and succinimide is separated out as a precipitate. Subsequently, the solution is refluxed for a further hour and cooled to room temperature. The precipitate is filtered off and washed with cyclohexane, and the organic phase is concentrated on a rotary evaporator under reduced pressure. 14.67 g (54.3 mmol, 92% of theory, 75% purity by NMR) of 3-(4-(bromomethyl)phenyl)-5,5-dimethyl-1,4,2-dioxazole were obtained as crude product, which can be converted further without further purification.

¹H NMR (400 MHz, chloroform-d) δ=7.95-7.90 (m, 2H), 7.20-7.14 (m, 2H), 4.12 (s, 2H), 1.67 (s, 6H).

4th Stage (4-(5,5-dimethyl-1,4,2-dioxazol-3-yl)benzyl)-triphenylphosphonium bromide

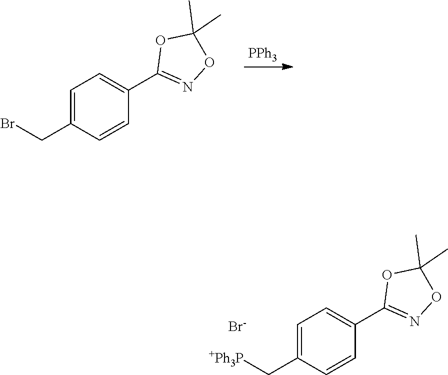

14.67 g (54.3 mmol, 1 eq) of 3-(4-(bromomethyl)-phenyl)-5,5-dimethyl-1,4,2-dioxazole are dissolved in 200 mL of dried acetone (3A molecular sieve). To this solution are added 14.24 g (54.3 mmol, 1 eq) of triphenylphosphine, and the solution is refluxed for 4 hours. Subsequently, the solution is concentrated to about ¼ of its total volume under reduced pressure, and 200 mL of diethyl ether are slowly added dropwise over a period of 2 hours. After cooling at −20° C., the precipitate is filtered off with suction, and 18.7 g (35.1 mmol, 65% of theory) of (4-(5,5-dimethyl-1,4,2-dioxazol-3-yl)benzyl)triphenylphosphonium bromide are obtained as a colorless solid.

¹H NMR (300 MHz, chloroform-d) δ 7.82-7.72 (m, 9H), 7.68-7.58 (m, 6H), 7.53-7.47 (m, 2H), 7.24-7.18 (m, 2H), 5.62 (d, J=15.0 Hz, 2H), 1.65 (s, 6H).

### 5th Stage 5,5-dimethyl-3-(4-vinylphenyl)-1,4,2-dioxazole

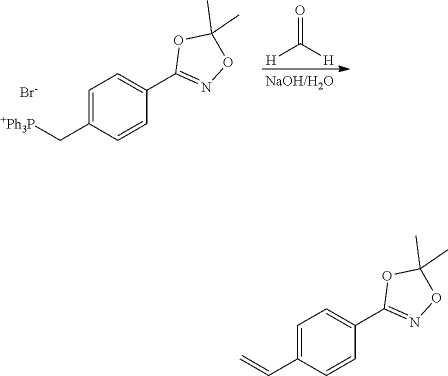

8.55 g (16.6 mmol, 1 eq) of (4-(5,5-dimethyl-1,4,2-dioxazol-3-yl)benzyl)triphenylphosphonium bromide are dissolved in 80 mL of aqueous formaldehyde solution (30%). Added dropwise to this solution are 5.31 g of NaOH in 24 mL of water within 20 minutes. The solution is stirred for 12 hours and extracted three times with 100 mL each time of dichloromethane. The combined organic phases are washed with 30 mL of saturated NaCl solution, dried over MgSO₄ and concentrated under reduced pressure. After purification by means of column chromatography (SiO₂, dichloromethane, R_{f}=1), 940 mg (4.6 mmol, 28% of theory) of 5,5-dimethyl-3-(4-vinylphenyl)-1,4,2-dioxazole were obtained as a colorless liquid.

¹H NMR (300 MHz, chloroform-d) δ=7.78-7.71 (m, 2H), 7.48-7.43 (m, 2H), 6.73 (dd, J=17.6, 10.9 Hz, 1H), 5.84 (dd, J=17.6, 0.7 Hz, 1H), 5.35 (dd, J=10.9, 0.7 Hz, 1H), 1.68 (s, 6H).

### EXAMPLE 5: METHACRYLATE DERIVATIVE WITH PROTECTED HYDROXAMIC ACID

### 2-(4-(5,5-dimethyl-1,4,2-dioxazol-3-yl)phenoxy)ethyl methacrylate

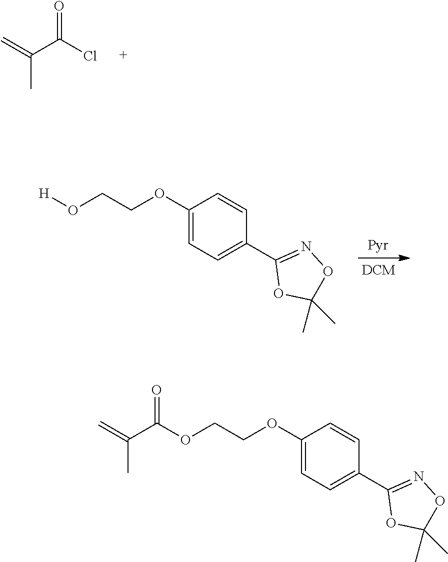

In a 5 mL Schlenk flask, 237 mg (1 mmol, 1 eq) of 2-(4-(5,5-dimethyl-1,4,2-dioxazol-3-yl)phenoxy)ethan-1-ol are dissolved in 2 mL of dichloromethane, and 97 μL (95 mg, 1.2 mmol, 1.2 eq) of pyridine are added. While cooling with ice, 107 μL (115 mg, 1.1 mmol, 1.1 eq) of methacryloyl chloride are added and the solution is stirred at room temperature for 1 h. After the solvent has been removed under reduced pressure, the residue is purified by column chromatography (SiO₂, PE/EA=4/1, Rf=0.5). 114 mg (0.37 mmol, 37% of theory) of 2-(4-(5,5-dimethyl-1,4,2-dioxazol-3-yl)phenoxy)ethyl methacrylate were obtained as a colorless liquid.

¹H NMR (300 MHz, chloroform-d) δ=7.76-7.66 (m, 2H), 6.99-6.88 (m, 2H), 6.15-6.12 (m, 1H), 5.62-5.57 (m, 1H), 4.54-4.46 (m, 2H), 4.29-4.22 (m, 2H), 1.96-1.93 (m, 3H), 1.66 (s, 6H).

### EXAMPLE 6: INITIATOR WITH PROTECTED HYDROXAMIC ACID FOR ANIONIC RING-OPENING POLYMERIZATION

### 1st Stage ethyl 4-(2-hydroxyethoxy)benzoate

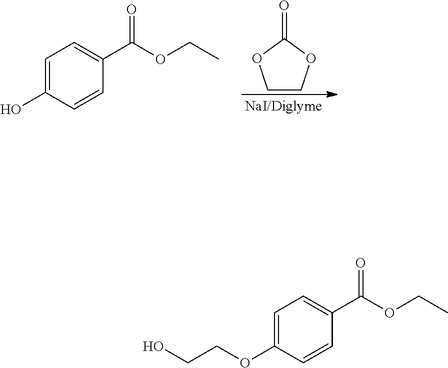

A 250 mL flask with reflux condenser and bubble counter is initially charged with 83.09 g (0.5 mol, 1 eq) of ethyl 4-hydroxybenzoate, 46.25 g (0.525 mol, 1.05 eq) of ethylene carbonate, 7.5 g (0.05 mol, 0.1 eq) of sodium iodide, and 100 mL of diglyme are added. The solution is boiled at 140° C. for 16 hours and then refluxed for 2 hours. After cooling to room temperature, the solution is completely freed of solvents under reduced pressure and the residue is dissolved in 300 mL of ethyl acetate. The organic phase is washed with 50 mL of water, 50 mL of saturated NaHCO₃ solution and 50 mL of saturated NaCl solution, dried over Na₂SO₄ and freed of the solvent under reduced pressure. The residue obtained is 100.2 g (0.477 mol, 95% of theory) of ethyl 4-(2-hydroxyethoxy)benzoate as a colorless solid.

¹H NMR (300 MHz, chloroform-d) δ=7.97-7.91 (m, 2H), 6.91-6.84 (m, 2H), 4.30 (q, J=7.1 Hz, 2H), 4.10-4.05 (m, 2H), 3.98-3.90 (m, 2H), 1.34 (t, J=7.1 Hz, 3H).

### 2nd Stage N-hydroxy-4-(2-hydroxyethoxy)benzamide

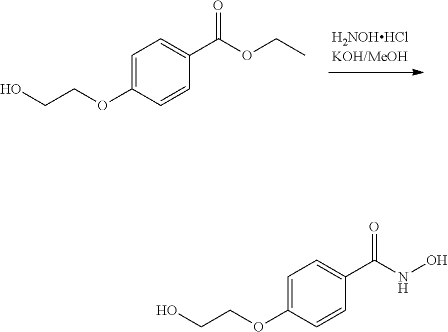

To a solution of 20.85 g (0.3 mol, 3 eq) of hydroxylamine hydrochloride in 300 mL of methanol are added, in multiple portions, 33.60 g (0.6 mol, 6 eq) of KOH, in such a way that the temperature remains below 45° C. Subsequently, 19.62 g (0.1 mol, 1 eq) of ethyl 4-(2-hydroxyethoxy)benzoate are added and the solution is stirred at room temperature overnight. The reaction mixture is subsequently acidified to pH 4 with concentrated hydrochloric acid and concentrated to dryness under reduced pressure. The residue is subjected to hot extraction under reflux in a Soxhlet apparatus with 500 mL of THF overnight. After cooling at −20° C., the product crystallizes out in the THF solution, and is filtered off with suction and washed with a little cold THF. 19.62 g (0.093 mol, 93% of theory) of N-hydroxy-4-(2-hydroxyethoxy)benzamide were obtained as colorless platelets.

¹H NMR (300 MHz, DMSO-d₆) δ=11.06 (s, 1H), 8.90 (s, 1H), 7.74-7.68 (m, 2H), 7.01-6.96 (m, 2H), 4.89 (t, J=5.5 Hz, 1H), 4.03 (t, J=4.9 Hz, 2H), 3.72 (dt, J=5.5, 4.9 Hz, 2H)

¹³C NMR (75 MHz, DMSO-d₆) δ=164.02, 160.95, 128.62, 124.82, 114.09, 67.70, 59.48.

### 3rd Stage 2- (4-(5,5-dimethyl-1,4,2-dioxazol-3-yl)-phenoxy) ethanol

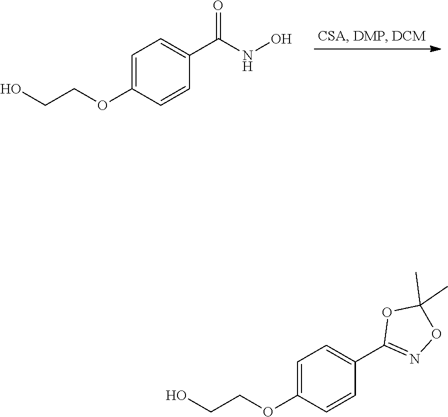 [SMILES: O=C(NO)c1ccc(OCCO)cc1 ; CC1(C)ON=C(c2ccc(OCCO)cc2)O1]

A dried 2 L round-bottom flask that has been repeatedly evacuated and filled with inert gas is initially charged with 5.0 g (25.35 mmol, 1 eq) of AT-hydroxy-4-(2-hydroxyethoxy)benzamide in 260 mL of dichloro-methane, and 9.35 mL (7.95 g, 76.31 mmol, 3 eq) of 2,2-dimethoxypropane (DMP) are added. Subsequently, 5.92 g (25.48 mmol, 1.05 eq) of DL-camphor-10-sulfonic acid (CSA) are added in one addition and the reaction mixture is stirred at room temperature for 5 h. Subsequently, the reaction is terminated by adding 850 mL of 2 molar aqueous NaOH solution and stirred at room temperature for 48 h. The organic phase is removed and the aqueous phase is extracted with 200 mL of dichloromethane. The combined organic phases are washed twice with 150 mL each time of 2 molar aqueous NaOH solution and once with 200 mL of saturated NaCl solution and dried over MgSO₄, and the solvent is removed completely under reduced pressure. The residue obtained was 2.45 g (10.33 mmol, 40% of theory) of 2-(4-(5,5-dimethyl-1,4,2-dioxazol-3-yl)phenoxy)ethanol as a colorless solid.

¹H NMR (300 MHz, DMSO-d₆) δ=7.74-7.68 (m, 2H), 6.99-6.94 (m, 2H), 4.12 (t, J=5.2 Hz, 2H), 3.98 (t, J=5.3 Hz, 2H), 1.66 (s, 6H)

¹³C NMR (75 MHz, DMSO-d₆) δ=161.12, 158.21, 128.58, 116.50, 115.34, 114.71, 61.43, 24.97.

### EXAMPLE 7: EPOXIDE MONOMER WITH PROTECTED HYDROXAMIC ACID GROUP

### 5,5-dimethyl-3-(4-(2-(oxiran-2-ylmethoxy)ethoxy)-phenyl)-1,4,2-dioxazole

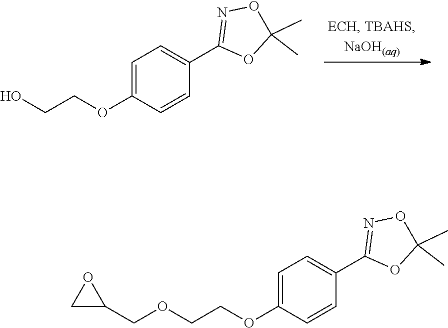 [SMILES: CC1(C)ON=C(c2ccc(OCCO)cc2)O1 ; CC1(C)ON=C(c2ccc(OCCOCC3CO3)cc2)O1]

A 3-neck sulfonation flask with precision glass stirrer, septum and thermometer with ground-glass joint was initially charged with 7 mL of 50% (w/w) aqueous NaOH. While cooling with ice, 146.7 mg (0.432 mmol, 0.04 eq) of tetrabutylammonium hydrogensulfate (TBAHS) and 4.3 mL (5.07 g, 54.83 mmol, 5.3 eq) of epichloro-hydrin are added. Subsequently added dropwise to this solution are 2.45 g (10.32 mmol, 1 eq) of 2-(4-(5,5-dimethyl-1,4,2-dioxazol-3-yl)phenoxy)ethanol in 10 mL of benzene within 25 minutes. After stirring at 10-15° C. overnight, the reaction mixture is added to 35 mL of ice/water mixture, and 25 mL of diethyl ether are added. The organic phase is removed and the aqueous phase is extracted twice with 50 mL each time of diethyl ether. The combined organic phases are washed with saturated NaCl solution to pH 7.0 (5× with about 100 mL of solution) and dried over Na₂SO₄. After the solvent has been removed under reduced pressure, the product is obtained by column chromatography purification (SiO₂, EA/PE=1/5). 2.02 g (6.9 mmol, 67% of theory) of 5,5-dimethyl-3-(4-(2-(oxiran-2-yl-methoxy)ethoxy)phenyl)-1,4,2-dioxazole were obtained as a colorless liquid.

¹H NMR (300 MHz, chloroform-d) δ=7.76-7.65 (m, 2H), 7.00-6.88 (m, 2H), 4.17 (dd, J=5.3, 4.4 Hz, 2H), 3.99-3.82 (m, 3H), 3.48 (dd, J=11.7, 6.0 Hz, 1H), 3.23-3.15 (m, 1H), 2.81 (dd, J=5.0, 4.1 Hz, 1H), 2.63 (dd, J=5.0, 2.7 Hz, 1H), 1.66 (s, 6H).

### EXAMPLE 8: BENZYL ALCOHOL-BASED INITIATOR

### 1st Stage N-hydroxy-4-(hydroxymethyl)benzamide

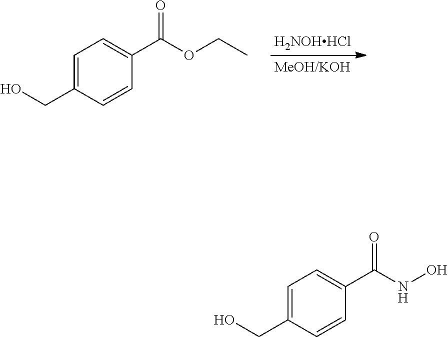

### Synthesis analogous to N-hydroxy-4-(2-hydroxyethoxy)-benzamide

Yield: 52% of theory.

¹H NMR (400 MHz, DMSO-d₆) δ=11.16 (s, 1H), 8.97 (s, 1H), 7.75-7.66 (m, 2H), 7.43-7.33 (m, 2H), 4.53 (s, 2H).

### 2nd (4-(5,5-dimethyl-1,4,2-dioxazol-3-yl)phenyl)methanol

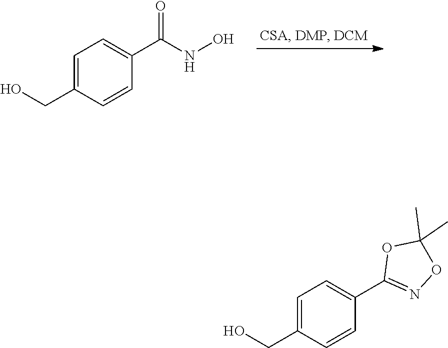

### Synthesis analogous to 2-(4-(5,5-dimethyl-1,4,2-dioxazol-3-yl)phenoxy)ethanol

Yield: 55% of theory.

¹H NMR (300 MHz, chloroform-d) δ=7.79-7.73 (m, 2H), 7.45-7.38 (m, 2H), 4.74 (s, 2H), 1.68 (s, 6H).

### EXAMPLE 9: EPOXIDE MONOMER FROM BENZYL ALCOHOL INITIATOR

### 5,5-dimethyl-3-(4-((oxiran-2-ylmethoxy)methyl)phenyl)-1,4,2-dioxazole

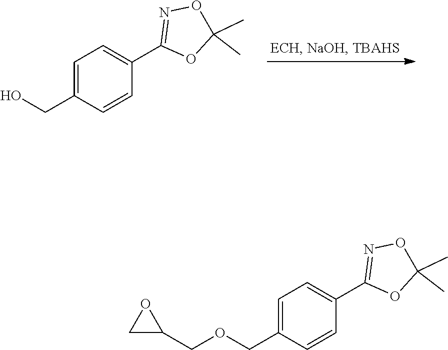 [SMILES: CC1(C)ON=C(c2ccc(CO)cc2)O1 ; CC1(C)ON=C(c2ccc(COCC3CO3)cc2)O1]

### Synthesis analogous to 5,5-dimethyl-3-(4-(2-(oxiran-2-ylmethoxy)ethoxy)phenyl)-1,4,2-dioxazole

Yield: 66% of theory.

¹H NMR (400 MHz, chloroform-d) δ=7.78-7.73 (m, 2H), 7.42-7.37 (m, 2H), 4.71-4.52 (m, 2H), 3.81 (dd, J=11.5, 2.8 Hz, 1H), 3.42 (dd, J=11.5, 5.9 Hz, 1H), 3.24-3.15 (m, 1H), 2.81 (dd, J=5.0, 4.1 Hz, 1H), 2.62 (dd, J=5.0, 2.7 Hz, 1H), 1.67 (s, 6H).

### EXAMPLE 10: ANIONIC POLYMERIZATION OF 5,5-DIMETHYL-3-(4-VINYLPHENYL)-1,4,2-DIOXAZOLE

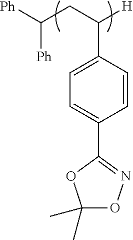

A 100 mL Schlenk flask is initially charged with 1.0 g (4.9 mmol, 10 eq) of 5,5-dimethyl-3-(4-vinylphenyl)-1,4,2-dioxazole dried azeotropically with 5 mL of benzene under high vacuum (1E-3 mbar) for 24 h. Subsequently, 10 mL of sodium-dried THF are distilled into the flask under protective gas and the solution is cooled to −78° C. by means of an acetone/dry ice bath. Initiation is effected by adding 1.0 mL (0.49 mmol, 1 eq) of a 0.5 molar diphenylmethylpotassium solution. After a reaction time of 2 h, the polymerization is terminated by adding 1 mL of methanol and freed of solvents under reduced pressure.

Mn (GPC, THF, PEG)=1280 g/mol PDI=1.4

### EXAMPLE 11: ANIONIC POLYMERIZATION OF EPOXY DERIVATIVES PROCEEDING FROM PROTECTED HYDROXAMIC ACIDS AS INITIATOR

A 100 mL Schlenk flask is initially charged with 0.043 mmol (1 eq) of initiator, and provided with 0.035 mmol (0.8 eq) of potassium Cert-butoxide and 0.070 mmol (1.6 eq) of 18-crown-6. Subsequently, for deprotonation, 1 mL of THF and 4 mL of benzene are added and the solution is stirred under static vacuum (about 500 mbar) at 40° C. for 30 minutes. The solvent is removed completely under high vacuum (10⁻³ mbar) and the initiator is dried for 24 h. Subsequently, the initiator is dissolved in 10 mL of dry THF (over sodium), provided with 23 mmol (40 eq) of dried monomer and polymerized at 40-60° C. for 24-48 h. Subsequently, the reaction is terminated by adding 1 mL of methanol, the solvents are removed under high vacuum and the polymer is purified by partitioning between dichloromethane and water. For this purpose, the residue is dissolved in dichloromethane and extracted by shaking with aqueous NaCl solution. The organic phase is removed and dried over Na₂SO₄ and freed completely of solvents under reduced pressure. Typical yields 80-95% of theory.

Reaction Conditions:

EO: no drying of the monomer, reaction time of 48 h at 60° C. under static vacuum (10⁻³ mbar)

PO: drying of the monomer over 3 A molecular sieve for 30 minutes, polymerization without solvent at 40° C. for 24 h under an argon atmosphere (1 atm).

### EXAMPLE 12: (4-(5,5-DIMETHYL-1,4,2-DIOXAZOL-3-YL)-PHENYL)METHANOL-INITIATED POLYPROPYLENE OXIDE

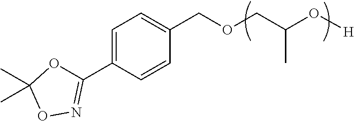

Batch: 1 eq of initiator, 20 eq of propylene oxide

¹H NMR (300 MHz, chloroform-d) δ=7.78-7.70 (m, 2H), 7.42-7.34 (m, 2H), 4.58 (s, 2H), 3.72-3.28 (m, 66H), 1.67 (s, 6H), 1.23-0.99 (m, 66H).

Mn (GPC, DMF, PEG)=1310 g/mol PDI=1.07

### EXAMPLE 13: 2-(4-(5,5-DIMETHYL-1,4,2-DIOXAZOL-3-YL)-PHENOXY)ETHANOL-INITIATED POLYPROPYLENE OXIDE

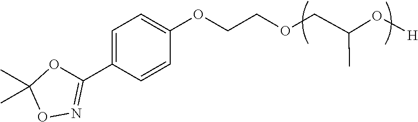

Batch: 1 eq of initiator, 20 eq of propylene oxide

¹H NMR (300 MHz, chloroform-d) δ=7.74-7.65 (m, 2H), 6.98-6.89 (m, 2H), 4.19-4.11 (m, 2H), 3.89-3.78 (m, 2H), 3.78-3.24 (m, 63H), 1.66 (s, 6H), 1.17-1.09 (m, 63H).

Mn (GPC, DMF, PEG)=1340 g/mol PDI=1.06

### EXAMPLE 14: (4-(5,5-DIMETHYL-1,4,2-DIOXAZOL-3-YL)-PHENYL)METHANOL-INITIATED POLYETHYLENE GLYCOL

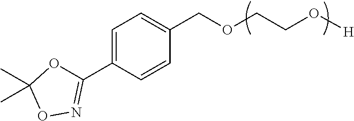

Batch: 1 eq of initiator, 50 eq of ethylene oxide ¹H NMR (300 MHz, chloroform-d) δ=7.84-7.71 (m, 2H), 7.47-7.37 (m, 2H), 4.62 (s, 2H), 3.82-3.52 (m, 203H), 1.70 (s, 6H).

Mn (GPC, DMF, PEG)=1340 g/mol PDI=1.06

### EXAMPLE 15: 2-(4-(5,5-DIMETHYL-1,4,2-DIOXAZOL-3-YL)-PHENOXY)ETHANOL-INITIATED POLYETHYLENE GLYCOL

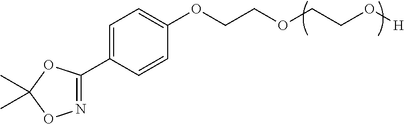

Batch: 1 eq of initiator, 50 eq of ethylene oxide

¹H NMR (300 MHz, chloroform-d) δ=7.72-7.65 (m, 2H), 6.97-6.88 (m, 2H), 4.19-4.11 (m, 2H), 3.91-3.81 (m, 3H), 3.75-3.54 (m, 284H), 1.66 (s, 6H).

Mn (GPC, DMF, PEG)=2050 g/mol PDI=1.07

### EXAMPLE 16: ANIONIC COPOLYMERIZATION OF EPOXY DERIVATIVES WITH EPOXIDE MONOMERS HAVING A PROTECTED HYDROXAMIC ACID GROUP

A 100 mL Schlenk flask is initially charged with 39.75 mg (0.165 mmol, 1 eq) of N,N-dibenzylaminoethanol (or benzyloxyethanol), 14.8 mg (0.14 mmol, 0.8 eq) of potassium tert-butoxide and 71.1 mg (0.269 mmol, 1.6 eq) of 18-crown-6, 1 mL of THF and 4 mL of benzene are added and the mixture is stirred under static vacuum (about 500 mbar) at 40° C. for 30 minutes. Subsequently, the initiator is freed of solvents without residue under high vacuum (10⁻³ mbar) and dried at 40° C. for 24 h. 10 mL of THF (dried over Na) are distilled into the flask and the dried monomers are added. After a reaction time of 48 h at 40-60° C., the polymerization is terminated by adding 1 mL of methanol and the solvent is removed without residue. The residue is taken up in dichloromethane and washed repeatedly with saturated sodium chloride solution. The organic phase is dried over Na₂SO₄, filtered and then freed of the solvent without residue under reduced pressure. The residue obtained is the polymer with typical yields of 80-95% of theory.

### EXAMPLE 17: RANDOM COPOLYMER OF PROPYLENE OXIDE AND 5,5-DIMETHYL-3-(4-((OXIRAN-2-YLMETHOXY)METHYL)PHENYL)-1,4,2-DIOXAZOLE

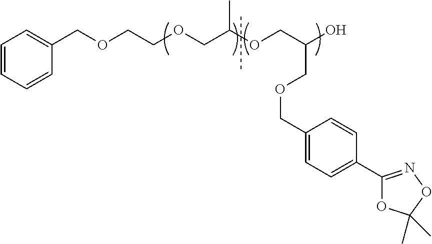

Conditions: initiator 1 eq (benzyloxyethanol), comonomer dried azeotropically using benzene under high vacuum (10⁻³ mbar) for h, PO dried over 3 A molecular sieve (30 minutes, RT), polymerization under argon atmosphere for 24 h.

¹H NMR (300 MHz, chloroform-d) δ=7.78-7.65 (m, 6H), 7.42-7.26 (m, 11H), 4.60-4.45 (m, 8H), 3.65-3.40 (m, 43H), 1.67 (s, 21H), 1.21-1.00 (m, 21H).

Comonomer content: 33 mol %

Mn (GPC, DMF, PEG)=918 g/mol PDI=1.25

### EXAMPLE 18: RANDOM COPOLYMER OF PROPYLENE OXIDE AND 5,5-DIMETHYL-3-(4-(2-(OXIRAN-2-YLMETHOXY)ETHOXY)-PHENYL)-1,4,2-DIOXAZOLE

Conditions: initiator 1 eq (N,N-dibenzylaminoethanol), comonomer dried azeotropically using benzene under high vacuum (10⁻³ mbar) for 24 h, PO dried over 3 A molecular sieve (30 minutes, RT), polymerization under argon atmosphere for 24 h.

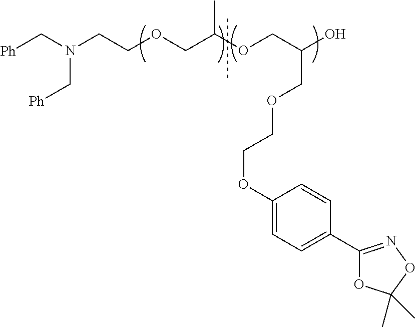

¹H NMR (400 MHz, chloroform-d) δ=7.73-7.64 (m, 4H), 7.39-7.33 (m, 4H), 7.32-7.27 (m, 2H), 7.24-7.16 (m, 2H), 6.97-6.80 (m, 5H), 4.14-3.94 (m, 2H), 3.79 (s, 4H), 3.71-3.27 (m, 58H), 1.66 (s, 12H), 1.18-1.05 (m, 47H).

Comonomer content: 15 mol %

Mn (GPC, DMF, PEG)=960 g/mol PDI=1.15

### EXAMPLE 19: RANDOM COPOLYMER OF ETHYLENE OXIDE AND 5,5-DIMETHYL-3-(4-((OXIRAN-2-YLMETHOXY)METHYL)PHENYL)-1,4,2-DIOXAZOLE

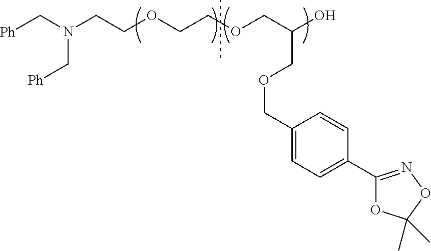

Conditions: 1 eq of initiator (N,N-dibenzylamino-ethanol), 50 eq of ethylene oxide, 5 eq of comonomer dried azeotropically using benzene under high vacuum (10⁻³ mbar) for 24 h, polymerization under reduced pressure at 60° C. for 48 h.

¹H NMR (400 MHz, chloroform-d) δ=7.80-7.62 (m, 10H), 7.43-7.31 (m, 18H), 4.62-4.38 (m, 3H), 3.92-3.26 (m, 287H), 2.72-2.59 (m, 2H), 1.74-1.63 (m, 28H).

Comonomer content: 7 mol %

Mn (GPC, DMF, PEG)=1920 g/mol PDI=1.08

### EXAMPLE 20: RANDOM COPOLYMER OF ETHYLENE OXIDE AND 5,5-DIMETHYL-3-(4-(2-(OXIRAN-2-YLMETHOXY)ETHOXY)PHENYL)-1,4,2-DIOXAZOLE

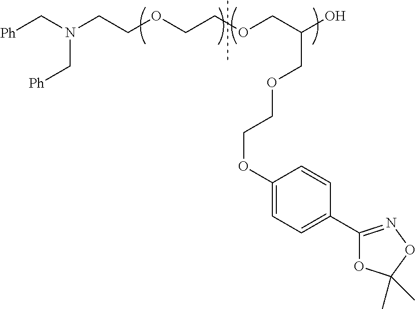

Conditions: 1 eq of initiator (N,N-dibenzylamino-ethanol), 50 eq of ethylene oxide, 10 eq of comonomer dried azeotropically using benzene under high vacuum (10⁻³ mbar) for 24 h, polymerization under reduced pressure at 60° C. for 48 h.

¹H NMR (400 MHz, chloroform-d) δ 7.76-7.62 (m, 10H), 7.40-7.32 (m, 4H), 7.00-6.85 (m, 10H), 4.21-4.11 (m, 10H), 3.89-3.79 (m, 10H), 3.78-3.49 (m, 202H), 2.71-2.60 (m, 2H), 1.67 (s, 26H).

Comonomer content: 10 mol %

Mn (GPC, DMF, PEG)=1830 g/mol PDI=1.13

### EXAMPLE 21: PREPARATION OF BLOCK COPOLYMERS FROM MPEG AND A PROTECTED HYDROXAMIC ACID BLOCK

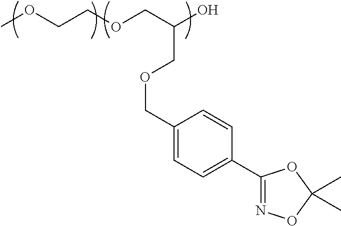

In a 50 mL Schlenk flask, 327.6 mg (0.1638 mmol, 1 eq) of polyethylene glycol monomethyl ether having an average molecular weight of 1600 g/mol (GPC, DMF, PEG, PDI=1.05), 14.5 mg (0.1292, 0.8 eq) of potassium Cert-butoxide and 70.2 mg (0.26 mmol, 1.6 eq) of 18-crown-6 are provided together with 1 mL of THF and 4 mL of benzene and stirred under static vacuum (about 50 mbar) at 60° C. for 60 minutes. Subsequently, any solvent is removed under high vacuum (10⁻³ mbar) and the initiator is dried at 80° C. for 24 h. The macroinitiator is dissolved in 5 mL of dry THF and provided with 200 mg (0.76 mmol, 4.6 eq) of 5,5-dimethyl-3-(4-((oxiran-2-yl-methoxy)methyl)phenyl)-1,4,2-dioxazole (dried azeotropically with benzene under high vacuum for 24 h) and stirred at 60° C. for 48 h. Subsequently, the reaction is terminated by adding 1 mL of methanol, the solvent is almost completely removed and the residue is precipitated in ice-cold diethyl ether. After centrifugation, the residue is taken up in dichloromethane and washed with saturated NaCl solution, and the organic phase is removed and dried over Na₂SO₄. After the solvent has been removed under reduced pressure, the product is obtained in a yield of 95% of theory.

¹H NMR (300 MHz, chloroform-d) δ=7.81-7.71 (m, 8H), 7.43-7.35 (m, 8H), 4.68-4.57 (m, 8H), 3.66-3.60 (m, 145H), 3.48 (s, 3H), 1.68 (s, 24H).

Comonomer content: 10 mol %

Mn (GPC, DMF, PEG)=2040 g/mol PDI=1.06

1. A process for preparing a pharmaceutical for physiological iron chelation, comprising the steps of
(a) providing an initiator selected from the group comprising
alcohols;
compounds containing a protected hydroxamic acid group selected from 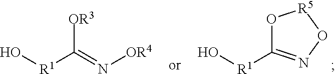
and
lithium organyls and free-radical initiators;
(b) providing at least one monomer selected from the group comprising
epoxides selected from 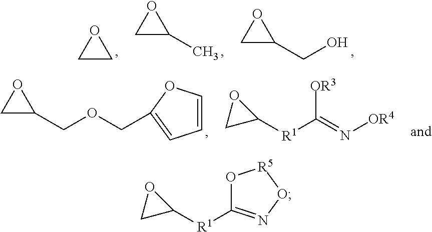
or
acrylics selected from 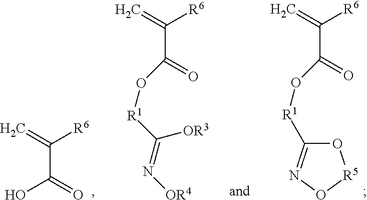
or
styrenes selected from 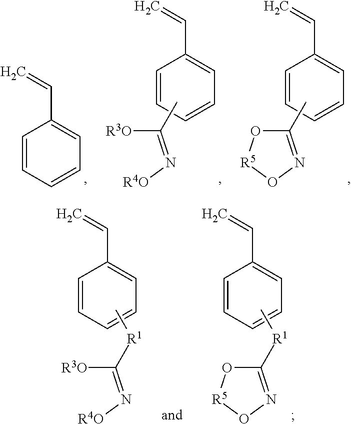
where
the initiator and/or one of the monomers contains a protected hydroxarnic acid group or at least one monomer is an epoxide including the epoxide 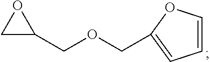 [SMILES: c1coc(COCC2CO2)c1]
R¹ is selected from
aliphatic groups having a formula —(CH₂)ₚ— with p=1, 2, 3, 4, 5, 6, 7, 8, 9 or 10;
alkoxide groups;
aliphatic ether groups having a formula —(CH₂)_{q}O(CH₂)ₛ— with q=1, 2, 3, 4, 5, 6, 7, 8, 9 or 10 and s=1, 2, 3, 4, 5, 6, 7, 8, 9 or 10;
oligoethylene glycol groups having the formula —(CH₂CH₂O)ₜ— with t=1, 2, 3, 4, 5, 6, 7, 8, 9 or 10;
aromatic groups; and
derivatives of the above groups;
R³ is a protecting group selected from the group comprising aliphatics —CₖH₂ₖ₊₁ with k=1, 2, 3, 4, 5, 6, 7, 8, 9 or 10, vinyl, allyl, phenyl, benzyl and silyls;
R⁴ is a protecting group selected from the group comprising aliphatics —CₘH₂ₘ₊₁ with m=1, 2, 3, 4, 5, 6, 7, 8, 9 or 10, vinyl, allyl, phenyl, benzyl and silyls;
R⁵ is selected from the group comprising ═(CH₂); acetonides; cyclohexanone radicals; and sodium tetraborate radical ═(B₄O₇);
R⁶ is selected from —H and —CH₃;
(c) mixing one or more of the monomers provided in step (b) with the initiator in a defined molar ratio to form a reaction mixture; and
(d) polymerizing the reaction mixture.

2. The process as claimed in claim 1, wherein the process further comprises adding, in a subsequent step (e), a further monomer listed in step (b) to the reaction mixture and continuing the polymerization.

3. The process as claimed in claim 2, wherein the process further comprises executing step (e) repeatedly.

4. The process as claimed in claim 2 wherein the process further comprises terminating the polymerizing step by consuming the at least one monomer or by addition of a terminator.

5. The process as claimed in claim 4, wherein the polymerization is ended by adding a terminator selected from the group comprising protic reagents; alcohols; alkyl halides; active esters; or activated carbonyl compounds.

6. The process as claimed in claim 4, wherein the at least one hydroxamic acid group is deprotected.

7. The process as claimed in claim 6, wherein the at least one hydroxamic acid group is deprotected by adding a deprotecting agent selected from the group comprising aqueous and nonaqueous solutions of inorganic acids; aqueous and nonaqueous solutions of organic acids; or by using acidic ion exchangers.

8. The process as claimed in claim 4, wherein said process further comprises including, in step (c) or (e), one or more of the epoxides listed in step (b) including the epoxide 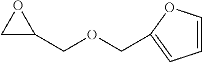 [SMILES: c1coc(COCC2CO2)c1]
and, after terminating the polymerization, adding a hydroxamic acid-functionalized compound having the formula 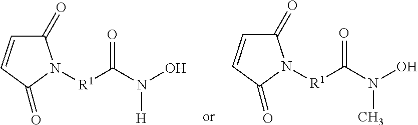
and conjugating the hydroxamic acid-functionalized compound with the furan groups of the polymer.

9. A pharmaceutical prepared by a process as claimed in claim 6.

10. A pharmaceutical comprising an initiator group, a polymer and an end group R⁷ and having an initiator group-polymer-R⁷ structure, which further comprises one or more functional hydroxamic acid groups having a formula —(C═O)NHOH or —(C═O)NCH₃OH type, wherein
the initiator group is selected from the group comprising
alkoxide groups;
hydroxamic acid-functionalized groups of the formula
—R¹(C═O)NHOH or —R³(C═O)NCH₃OH; and
residues of a lithium organyl or free-radical initiator; and
the polymer consists of units selected from the group comprising 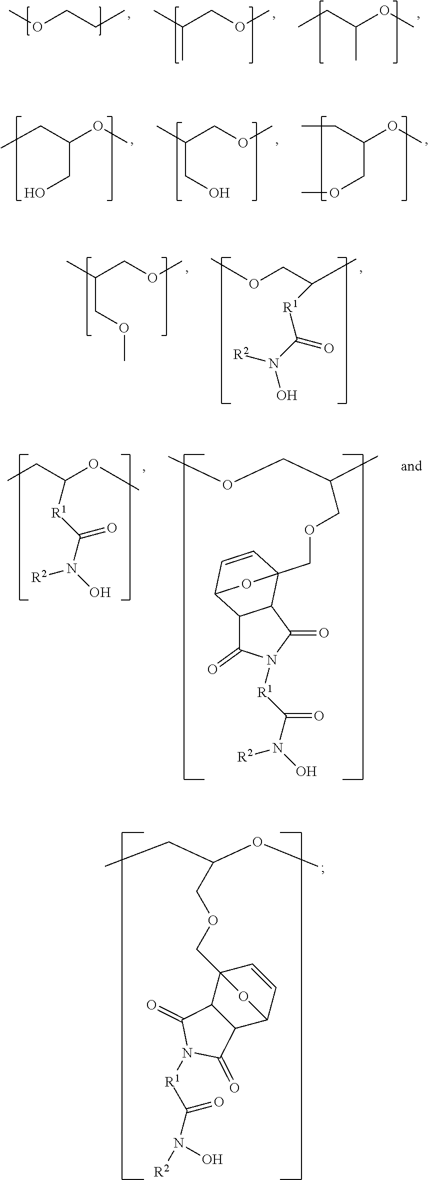
or
the polymer consists of acrylic units selected from the group comprising 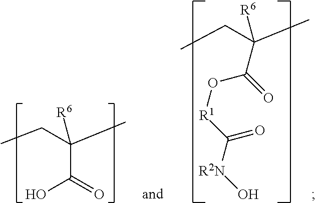
or
the polymer consists of styrene units selected from the group comprising 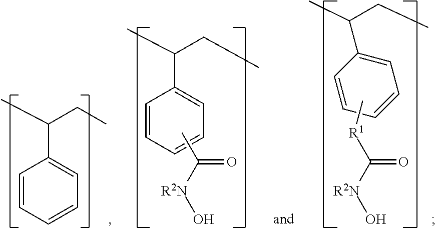
where
R¹ is selected from
aliphatic groups having a formula —(CH₂)ₚ— with p=1, 2, 3, 4, 5, 6, 7, 8, 9 or 10;
alkoxide groups;
aliphatic ether groups having a formula —(CH₂)_{q}O(CH₂)ₛ— with q=1, 2, 3, 4, 5, 6, 7, 8, 9 or 10 and s=1, 2, 3, 4, 5, 6, 7, 8, 9 or 10;
oligoethylene glycol groups having a formula —(CH₂CH₂O)ₜ— type with t=1, 2, 3, 4, 5, 6, 7, 8, 9 or 10;
aromatic groups; and
derivatives of the above groups;
R² is selected from —H and —CH₃;
R⁶ is selected from —H and —CH₃;
R⁷ is selected from the group comprising —H; —CH₃;
—(CH₂)ᵤCH₃ with u=1, 2, 3, 4, 5, 6, 7, 8, 9 or 10; esters; allyl radicals; propargyl radicals; and alcohol radicals.

11. The pharmaceutical as claimed in claim 10, wherein R¹ is a pentanol group —O(CH₂)₅— or a phenol group —O(C₆H₄)—.

12. The pharmaceutical as claimed in claim 10, wherein the pharmaceutical has a structure of either OHNH(C═O)R¹-polymer-R⁷ or OHNCH₃(C═O)R¹-polymer-R⁷, and the polymer is a polyethylene glycol —(CH₂CH₂O)ₙ— with 3≤n≤100.

13. The pharmaceutical as claimed in claim 10 to 12, wherein the pharmaceutical has a polydispersity M̅_{w}/M̅ₙ≤2.

14. The pharmaceutical as claimed in claim 13, wherein the pharmaceutical has a polydispersity M̅_{w}/M̅ₙ≤1.6.

15. The pharmaceutical as claimed in claim 10, wherein the pharmaceutical has a molar mass MW, with 600 g·mol⁻¹≤MW≤40 000 g·mol⁻¹.

16. The pharmaceutical as claimed in claim 15, wherein the pharmaceutical has a molar mass MW of 800 g·mol⁻¹≤M≤40 000 g·mol⁻¹.

17. The process as claimed in claim 1, wherein
(i) the alcohols are selected from HOCH₃, HOCH₂CH₃, HO(CHCH₃)CH₃, HO(CH₂)₂CH₃, HO(CH₂)₃CH₃, and HO(CH₂)₄CH₃;
(ii) the lithium organyls and free-radical initiators are selected from n-butyilithium, sec-butyllithiurn, dibenzoyl peroxide, azoisobutyronitile, potassium peroxodi-sulfate, and ammonium peroxodisulfate;
(iii) the R¹ alkoxide group is selected from —OCH₂—, —OCH₂CH₂—, —O(CHCH₃)CH₂—, —O(CH₂)₃—, —O(CH₂)₄—, and —O(CH₂)₅—; and the
R¹ aromatic groups are phenol or naphthyl radicals; and
(iii) the R³ and R⁴ silyls are trimethylsilyl or triisopropylsilyl; and
(iv) the R⁵ acetonides are ═(C(CH₃)₂) or ═(CHPh) and the cyclohexanone radicals are ═(C₆H₁₀).

18. The process as claimed in claim 5, wherein the protic reagent is H₂O; the alcohol is methanol, ethanol or propanol; the alkyl halide is methyl iodide, ethyl bromide, allyl chloride, allyl bromide, propargyl bromide; and the activated carbonyl compound is acid chloride, acid anhydride or N-hydroxysuccinimide ester.

19. The process as claimed in claim 7, wherein the inorganic acid is hydrochloric acid or sulfuric acid; and the organic acid is para-toluenesulfonic acid or camphor-10-sulfonic acid.

20. A pharmaceutical as claimed in claim 10, wherein
(i) the alkoxide group is —OCH₃, —OCH₂CH₃, —O(CHCH₃)CH₃, —O(CH₂)₂CG₃, —O(CH₂)₃CH₃, or —O(CH₂)₄CH₃; and
(ii) the residue of the lithium organyl or free-radical initiator is CH₃ (CH₂)₃—, CH₃CH₂(CHCH₃)—, Ph(C═O)O—, CNCH₃CH₃C—, or SO₂OHO—;
(iii) the R¹ alkoxide group is —OCH₂, —OCH₂CH₂—, —O(CHCH₃)CH₂—, —O(CH₂)₃—, —O(CH₂)₄—, or —O(CH₂)₅—; the R¹ aromatic groups are phenol or naphthyl radicals
(iv) the R⁷ esters are —(C═O)CH₃ and the R⁷ alcohol radicals are —OCH₃; —OCH₂CH₃; —OCH(CH₃)₂; or —O(CH₂)₂CH₃.

21. The pharmaceutical as claimed in claim 14, wherein the pharmaceutical has a polydispersity M̅_{w}/M̅_{b}≤1.2.

22. The pharmaceutical as claimed in claim 14, wherein the pharmaceutical has a polydispersity M̅_{w}/M̅ₙ≤1.1.

23. The pharmaceutical as claimed in claim 15, wherein the pharmaceutical has a molar mass MW of 1000 g·mol⁻¹≤MW≤40 000 g·mol⁻¹.
